(19) United States
(12) Patent Application Publication
(10) Pub. No.: US 2008/0108895 A1
(43) Pub. Date: May 8, 2008

(54) METHOD AND SYSTEM FOR DEFINING AT LEAST ONE ACQUISITION AND PROCESSING PARAMETER IN A TOMOSYNTHESIS SYSTEM

(75) Inventors: John Michael Sabol, Sussex, WI (US); Timothy Wayne Deller, Waukesha, WI (US); Kadri Nizar Jabri, Waukesha, WI (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/556,978

(22) Filed: Nov. 6, 2006

(51) Int. Cl. A61B 6/03; G06K 9/46

(52) U.S. Cl. 600/425; 378/26; 382/131

(57) ABSTRACT

A method and system for defining at least one of a plurality of acquisition and processing parameters in a tomosynthesis imaging system are disclosed herein. The method involves providing a user interface that allows a user to quickly and easily specify at least one desired characteristic of a reconstructed image. Based on the user-specified image characteristic, at least one of a desired set of acquisition and processing parameters for the tomosynthesis imaging system is automatically defined. The user interface interacts with a processor for deriving acquisition and processing parameters based upon image characteristics specified by the user using a user interface.

### FIELD OF THE INVENTION

This invention generally relates to an imaging system, and more particularly to methods and systems for defining at least one of the acquisition and processing parameters in a tomosynthesis system.

### BACKGROUND OF THE INVENTION

In classical tomography, the X-ray source and detector move synchronously and continuously in opposite directions about a pivot point residing in the plane of interest. The tomography procedure produces an image, or tomogram, of the desired plane by blurring the contributions from other planes. Digital tomosynthesis (DTS) is a limited angle imaging technique, which allows the reconstruction of tomographic planes on the basis of the information contained within the images acquired during one tomographic image acquisition. A set of two-dimensional (2-D) images of the object is obtained, each at a different projection angle, and a three-dimensional (3-D) image is generated from the same. For generating 3-D images, normally back projection techniques are used. Digital tomosynthesis is a new imaging technique that enables 3-D imaging of the patient using a large-area digital detector typically used for digital radiography. 3-D data is generated in the form of a number of slices through the patient, each parallel to the detector plane. The acquisition consists of a number of projections covering an angular range less than 180 degrees, typically 20 to 40 degrees.

The benefits of tomosynthesis imaging are well known theoretically, and applications such as breast tomosynthesis are clearly identified. For other body parts, however, neither physicists nor radiologists have a complete understanding of the possible clinical applications for this new imaging technique. As a result, it is likely that there will be an extended period of experimentation during which clinicians, physicists, and engineers will be examining new clinical applications both in the lab and in the clinic. Due to the increased complexity of the acquisition, the number of parameters that need to be specified for a tomosynthesis image acquisition is considerable. In addition to all of the same parameters which can be adjusted for a standard radiographic examination (e.g., kVp, mA, exposure time, collimation, field-of-view, dose, post-acquisition image processing, etc.), tomosynthesis requires specification of a number of acquisition and processing parameters unique to tomosynthesis (e.g., the number of projections, dose per projection, sweep angle, total dose, angular increment between projections, reconstruction algorithm, reconstruction ‘kernel’ or filter, etc.). All of these parameters have significant effect on the nature of the reconstructed slices including noise, slice thickness (z-resolution), prevalence of ripple artifacts, focal depth, field-of-view, number of slices that need to be read, etc. As the complexity involved is quite evident, there exists a need to provide a simple tool that will allow a user or an operator of the tomosynthesis system to select the desired acquisition and processing parameters based on clinical requirements without the need to understand physics and geometrical complexities of the tomosynthesis technique.

Thus it would be desirable to provide a user interface, which allows the user to indirectly select the desired acquisition and processing parameters without needing to understand or become involved in the complexities of the tomosynthesis technique.

### SUMMARY OF THE INVENTION

The above-mentioned shortcomings, disadvantages and problems are addressed herein which will be understood by reading and understanding the following specification.

The present invention provides a method of defining at least one of a plurality acquisition and processing parameters in a tomosynthesis imaging system. The method includes the steps of: providing a user interface for allowing a user to specify at least one characteristic of a reconstructed image; and defining at least one of a plurality of acquisition and processing parameters in the tomosynthesis system based on at least one image characteristic specified using the user interface. In an embodiment the user interface is configured to be a visual interface, which will allow a user to select one or more image characteristics. The user interface interacts with a processor for deriving desired acquisition and processing parameters based on the user-specified image characteristics. In an embodiment the image characteristics include both the characteristics of reconstructed image and anatomic characteristics.

In another embodiment, a tomosynthesis system with a user interface for allowing the user to indirectly select at least one of a plurality of acquisition and processing parameters is provided. The system comprises: an imager for providing images; and a computer including: a user interface for allowing a user to specify at least one characteristic of a reconstructed image; and a processor coupled to the user interface, the processor being programmed to define at least one of a plurality of acquisition and processing parameters for the imager based on at least one user-specified image characteristics.

In yet another embodiment a computer program, provided on one or more computer readable media for selecting at least one of plurality of acquisition parameters in a tomosynthesis imaging system is disclosed. The computer programincludes: a routine for providing a user interface for allowing a user to select at least one characteristic of a reconstructed image; and a routine for defining at least one of a plurality of acquisition and processing parameters based on the user-specified image characteristic. The routine for defining at least one of a plurality of acquisition and processing parameter comprises: a routine for obtaining at least one image characteristic from the user, the image characteristics include both characteristics of reconstructed image and anatomic characteristics. The routine for defining at least one of acquisition or processing parameter further comprises: a routine for deriving at least one of acquisition and processing parameters using a data base which stores relations between the image characteristics with acquisition parameters and processing parameters.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a method of tomosynthesis in accordance with an embodiment of the present invention;

FIG. 2 is a schematic diagram illustrating a tomosynthesis system which is capable of implementing a user interface as described in an embodiment of the invention;

FIG. 3 is a flowchart illustrating the exemplary steps of selecting desired acquisition and processing parameters as described in an embodiment of the invention;

FIG. 4 shows a reconstructed image illustrating the effects of rippling artifacts in a reconstructed image;

FIGS. 5A and 5B show reconstructed images depicting the relationship between rippling artifacts and thickness of body part being imaged;

FIGS. 6A and 6B show reconstructed images depicting the relationship between sweep angle and slice thickness;

FIGS. 7A, 7B, 7C and 7D show reconstructed images depicting the relationship between projection density, sweep angle and ripple artifacts;

FIGS. 8A and 8B show reconstructed images depicting the relationship between x-ray dose and noise artifacts; and

FIG. 9 shows an example of a user interface as described in an embodiment of the invention.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

In various embodiments, a method of defining at least one of a plurality of acquisition and processing parameters in tomosynthesis imaging system is provided. This is achieved by providing a user interface that is programmed to define at least one of desired acquisition and processing parameters based on at least one characteristic of a reconstructed image specified by a user using the user interface. It should be noted that the method and system described hereinafter is capable of defining at least one of an acquisition parameter or processing parameter, or a combination of both types of parameters, based on one or more user-specified image characteristics.

While the present technique is described herein with reference to particular tomosynthesis imaging applications, it should be noted that the invention is not limited to this or any particular application or environment. Rather, the technique may be employed in any digital tomosynthesis devices and in a range of applications, such as breast imaging, Chest radiography, baggage and parcel handling and inspection, part inspection and quality control, and so forth, to mention but a few.

In an embodiment the invention provides a tomosynthesis system, which allows a user to specify at least one of the acquisition or processing parameters. The acquisition and processing parameters are selected automatically based on the image characteristics, which can be specified by a user with the help of a user interface provided. In an embodiment the image characteristics include both characteristics of the reconstructed image and anatomic characteristics specific to a patient or an exam.

In different embodiments the invention provides a tool that will be used for determining at least one of a plurality of acquisition and processing parameters for a desired tomosynthesis scan. The user will be prompted for information of the anatomy to be scanned and desired output image characteristics. The user can also specify the level of importance of each of these desired output characteristics. Based on this information, the tool will compute an optimal set of at least one of imaging acquisition and processing parameters for the specific application. In an embodiment the invention provides a method for selecting at least one of the desired acquisition and processing parameters in a tomosynthesis system based on desired image characteristics, specified by a user.

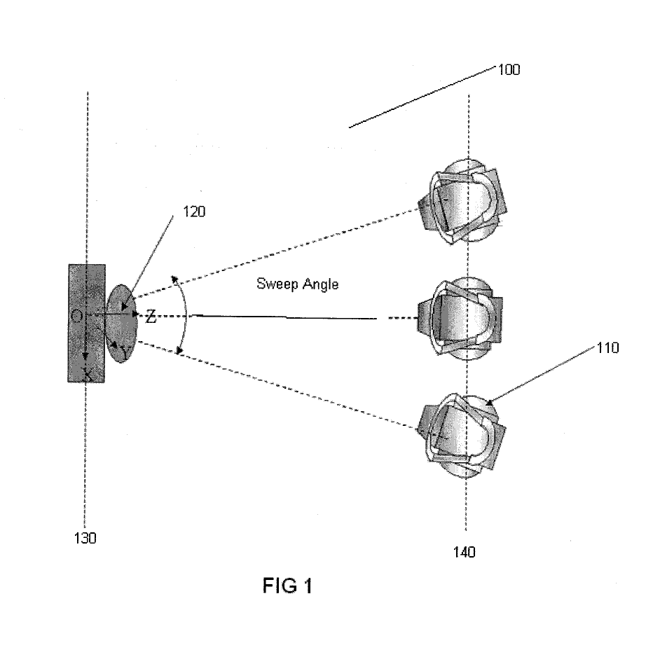

FIG. 1 illustrates a schematic diagram illustrating a method of tomosynthesis in accordance with an embodiment of the present invention. Tomosynthesis is an X-ray radiographic advanced imaging application that allows retrospective reconstruction of an arbitrary number of tomographic planes of an object from a set of low-dose projection images acquired over a limited angle. Digital tomosynthesis is a reconstruction of three-dimensional (3-D) images from two-dimensional (2-D) projection images of an object. The digital tomosynthesis system 100 comprises an X-ray source 110 and a 2-D X-ray detector 130, which is a digital detector. The object 120, being imaged is placed between the source 110 and the detector 130. In typical digital tomosynthesis systems, during data acquisition, the X-ray source 110 is rotated by a gantry (not shown) on an arc through a limited angular range about a pivot point and a set of projection radiographs of the object are acquired by the detector 130 at discrete locations of the X-Ray source 110. During the acquisition, the X-ray source 110 travels along the direction illustrated in FIG. 1, and rotates in synchrony such that the X-ray beam always point to the detector during the acquisition. The detector is maintained at a stationary position as the radiographs are acquired. Furthermore, the source 110 may be moved, typically within a focal spot plane 140 (although it may be moved outside of a single plane), which is substantially parallel to the detector 130. A plurality of radiographic views from different view angles may thus be collected by the detector 130.

In one embodiment a single source is provided, and the X-ray source delivers multiple exposures during a single “sweep” from multiple projection angles. The patient stands near the detector plane during the tomosynthesis scan. The number of projections for a single wallstand scan will range from about 30 to 60. The sweep angle is the angle from the first to the final projection focal spot with respect to the focal spot plane, and it will typically range from 30 to 50 degrees. It should be noted that a particular application may include different numbers of projections, including fewer than 30 or more than 60. It will also be noted that different sweep angles may be used.

The detector 130 is generally formed by a plurality of detector elements, generally corresponding to pixels, which sense the intensity of X-rays that pass through and around a region of interest. Depending upon the X-ray attenuation and absorption for the intervening structures, the radiation impacting each pixel region will vary. Each detector element produces an electrical signal that represents the intensity of the X-ray beam at the position of the element on the detector.

Once the projection radiographs have been obtained, they are then spatially translated with respect to each other and superimposed in such a manner that the images of structures in the tomosynthesis plane overlap exactly. The images of structures outside the tomosynthesis plane do not overlap exactly, resulting in a depth dependent blurring of these structures. By varying the amount of relative translation of the projection radiographs, the location of the tomosynthesis plane can be varied within the object. Each time the tomosynthesis plane is varied, the image data corresponding to the overlapping structures is superimposed and a 2-D image of the structure in the tomosynthesis plane is obtained. Once a complete set of 2-D images of the object has been obtained, a 3-D image of the object is generated from the set of 2-D images.

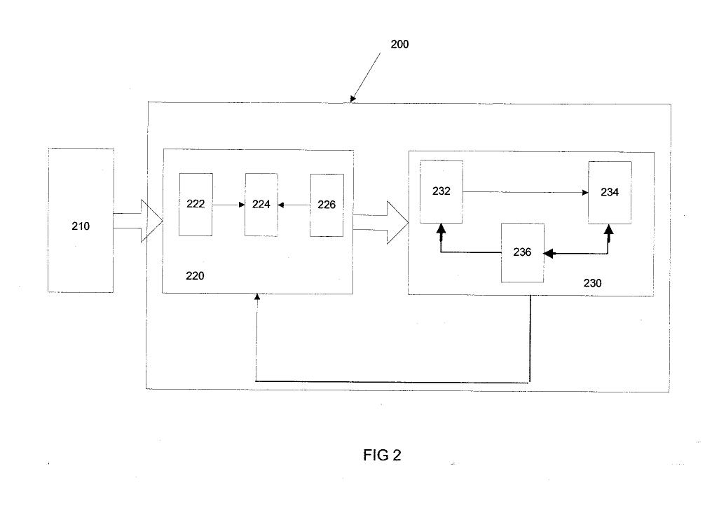

FIG. 2 illustrates diagrammatically an imaging system 200 capable of implementing a user interface for selecting at least one acquisition and/or processing parameter as described in an embodiment of the invention. The imaging system 200 may be used for acquiring and processing projection image data and reconstructing a volumetric image or 3-D image representative of the imaged object. In the illustrated embodiment, the imaging system 200 is a tomosynthesis system designed both to acquire projection image data, and to process the image data for display and to analyze the effect of various acquisition parameters in the quality of reconstructed images in accordance with the present technique. In the embodiment illustrated in FIG. 2, a user or an operator 210 interacts with the tomosynthesis system 200 for operating the same. The tomosynthesis system 200 includes a computer 220 and an imager 230.

In an embodiment the computer 220 is designed to enable rapid selection of parameters for tomosynthesis acquisition and processing by enabling a translation of the desired clinically relevant image characteristics for the desired application into the underlying parameters that control the tomosynthesis system. Through extensive characterization of the performance of the tomosynthesis system, the desired specifications of the reconstructed images can be translated into the required acquisition and/or processing parameters. In an embodiment this technique is implemented through the use of a software tool or algorithm which would define or influence the required acquisition parameters such as sweep angle, number of projections, and dose per projection and/or processing parameters such as reconstruction filter, slice pitch, edge enhancement, noise reduction, number of reconstructed images, averaging or combining of reconstructed images.

For achieving the above mentioned features the computer 220 is provided with a user interface 222 for interacting with the user 210 for selecting the desired acquisition and/or processing parameters. The user interface 222 is a visual interface that allows the user 210 to select at least one characteristic of a reconstructed image. The image characteristics include characteristics of the reconstructed image such as slice thickness, ripple artifacts, image noise level, motion artifacts or field of view, and anatomic characteristics such as body part thickness, high contrast contents creating ripple artifacts, anatomy density or scan orientation, but a person of skill in the art will understand that the image characteristics may not be limited to these. The anatomic characteristics may be patient or exam specific. The computer 220 further comprises a processor 224 for deriving the acquisition and/or processing parameters based on the user-specified image characteristics. The processor 224 is further provided with a memory 226 for storing a database. The database stores various relations between the image characteristics with acquisition and/or processing parameters. The user interface 222 is further configured to interact with the processor 224 for deriving the desired acquisition and/or processing parameter based on the relations stored in the data base and the image characteristics received from the user interface 222. Various acquisition parameters may include X-ray source sweep angle, number of projections, dose per projection, total dose, X-ray exposure time or collimation, and the processing parameters may include reconstruction filter, slice pitch, edge enhancement, noise reduction, number of reconstructed images, averaging or combining of reconstructed images.

The imager 230 includes a source of radiation 232, a detector 234 and a controlling device 236. The source of radiation 232 typically produces X-ray radiation in tomosynthesis; the source 232 is freely movable relative to the imaged object. In this exemplary embodiment, the X-ray radiation source 232 typically includes an X-ray tube and associated support and filtering components. In certain systems, however, more than one source of radiation may be employed. A stream of radiation emitted by the source 232 impinges an object (not shown) for example, a patient in medical applications. A portion of the radiation passes through or around the object and impacts a detector 234. The detector 234 comprises an array of detector elements, which produces electrical signals that represent the intensity of the incident X-ray beam. These signals are acquired and processed to reconstruct a volumetric image or 3-D image of the features within the object.

In one embodiment, the detector 234 is an amorphous silicon flat panel digital X-ray detector. However, the detector 234 may be any X-ray detector that provides a digital projection image including, but not limited to, a charge-coupled device (CCD), a digitized film, or another digital detector such as a direct conversion detector. In an embodiment the output of the detector may be fed to the computer 220 for processing the plurality of signals received from the detector to generate a plurality of projection images.

The source 232 is controlled by a controlling device 236 which furnishes both power and control signals for tomosynthesis examination sequences, including positioning of the source 232 relative to the object and the detector 234. Moreover, detector 234 is coupled to the controlling device 236, which commands acquisition of the signals generated in the detector 234. The controlling device 236 may also execute various signal processing and filtration functions, such as for initial adjustment of dynamic ranges, interleaving of digital image data, and so forth. In general, controlling device 236 commands operation of the imaging system 200 to execute examination protocols and to process acquired data.

In an embodiment, controlling device 236 may also include signal processing circuitry, typically based upon a general purpose or application-specific digital computer, associated memory circuitry for storing programs and routines executed by the computer, as well as configuration parameters and image data, interface circuits, and so forth.

In an embodiment the controlling device 236 receives instructions from the computer 220. The processor 224 of the computer 220 will select the desired acquisition and/or processing parameters based on the user-specified image characteristics, received through the user interface 222. Based on the defined acquisition and/or processing parameters, the computer 220 will send instructions to the controlling device 236. Based on the instruction received, the controlling device 236 will control the source 232 or detector 234 for achieving the desired acquisition and/or processing parameters. The controlling device 236 may control the orientation of the source, exposure time, collimation, field of view, dose per projection, total of dose of exposure, number of projections, angular increment between projections, sweep angle etc, but need not be limited to this.

In an embodiment the tomosynthesis system is provided with a collimator to minimize the radiation exposure to the object being imaged. A collimator (not shown) may be placed before or after the patient or object on need basis. Generally in digital tomosynthesis pre-patient collimation is used. The collimator may define the size and shape of the X-ray beam that emerges from the X-ray source. Apparently, the collimator defines the field-of-view (FOV) in the projection images so that unnecessary radiation can be avoided as much as possible. The controlling device 236 may control the operation of the collimator for controlling the field-of-view of the image and the collimation effects on the image, based on the instructions received from the computer 220.

In an embodiment the controlling device 236 based on the instructions received from the computer 220 may control the detector for controlling nature of the reconstructed slices including noise, slice thickness (z-resolution), prevalence of ripple artifacts, focal depth, field-of-view, number of slices that need to be read or the appropriate reconstruction algorithms.

In an embodiment the user interface 222 may be provided as an integral part of the controlling device 236.

In an embodiment the imager 230 is coupled to the computer 220. The computer 220 may act as a controlling device for controlling operation of the imager 230. The computer 220 may generate the control signals directly to control the operation of the imager 230, without using the controlling device 236.

In an embodiment, the user interface 222 is a visual interface, which will allow the user to select the listed image characteristics. The visual interface can display different image characteristics, anatomic characteristics etc. In an embodiment the user interface may be configured to have some predefined templates. For example if the user does not want to select any image characteristics, there exist some desired templates for different anatomy and/or for different image characteristics, so that the user can select one of the templates available on the visual interface. For example, if the user is going to take the image of a hand, there can be some standard templates for hand, which the user can select, if the user does not have any other specific requirements. In an embodiment the user interface has a plurality of interface keys for selecting image characteristics, anatomic characteristics, and acquisition and/or processing parameters. The interface keys are soft keys such as touch screen display or buttons and may be configured to appear automatically on the visual interface upon frequent use.

In an embodiment memory 226 of the processor 224 has a database stored with various relation of the image characteristics and/or anatomic characteristics with the acquisition and processing parameters. The database includes the complex interactions between the different image characteristics and the acquisition and processing parameters and have been established using a theoretical analysis and a large set of experiments on non-humanoid and humanoid phantoms. The processor 224 is also configured to derive the desired acquisition based on the instructions received from the computer 220 parameters based on the relations stored in the database and the desired image characteristics received from the user interface 222 in response to user actuations.

In an embodiment the user interface 222 will interact with the processor 224 through use of an algorithm that places weight on the output characteristics of image and their importance in order to balance the acquisition or processing parameter tradeoffs. For example, if suppression of a ripple artifact were more important than a narrow slice thickness for a particular application, then the back end would compute a smaller sweep angle. If low image noise is extremely important, then dose would be increased. If the anatomy has a small total thickness (for example, a wrist or a hand has a smaller body part thickness than a chest), then fewer projections would be used.

Some of the image characteristics, anatomic characteristics and acquisition parameters which may be used in tomosynthesis system and their inter relations are described in tabular form as shown below:

| “Definitions” | | |
|---|---|---|
| means Image | “Definitions” means | “Definitions” means |
| characteristic | Anatomic characteristic | Acquisition characteristic |
| Slice thickness | n/a | Sweep angle |
| Noise level | Tissue density, patient | Dose (kV, mA, Xray |
| | orientation, body part | exposure time) |
| | thickness | |
| Ripple artifact | Body part thickness, high- | Projection density (# of |
| level | contrast interfaces | projections/sweep angle) |
| Motion artifacts | Ability to keep anatomy | Time of scan (determined |
| | stationary (e.g.:, chest more | primarily by number of |
| | difficult than wrist) | projections) |
| Field of View/ | Size & shape of anatomy to | n/a |
| Collimation | be scanned | |

In an embodiment the user interface 222 is provided with an option of selecting various anatomic characteristics. The anatomic characteristics may be selected manually by the user. Alternately the anatomic characteristics may be selected from the various templates provided on the user interface 222. Also in an embodiment the user interface 222 may detect the object to be imaged and may automatically select the anatomic characteristics of the object.

In an embodiment the user interface 222 may be provided with a list of acquisition parameters, which may be specified by the user without actually interacting with a database. For example, if an experienced radiologist wants to specify some acquisition parameter without specifying image characteristics, he may select the required acquisition parameters from the front end of the user interface.

In an embodiment the user interface 222 may be provided with a list of processing parameters, which may be specified by the user without actually interacting with the database. For example, if an experienced radiologist wants to specify some processing parameter without specifying image characteristics, he may select the required processing parameters from the front end of the user interface. The processing parameters may include reconstruction filter, slice pitch, edge enhancement, noise reduction, number of reconstructed images, averaging or combining of reconstructed images.

In an embodiment the user is allowed to select the acquisition parameters during the acquisition. This is achieved by using the user interface as an “on the fly” tool, whereby the user/clinician is faced with a new clinical condition or scenario and would like to optimize the tomosynthesis acquisition based on his/her expectations of the required image characteristics. However the acquisition parameters are selected before acquisition of each slice of image.

In an embodiment the user specifies at least one image characteristic as a specific value or as a value within a range of desired values. Also the user may specify relative importance or significance of a plurality of image characteristics.

In an embodiment an input to the user interface is captured and annotated to a resulting image and is available for display to the user. In another embodiment, the defined acquisition and processing parameters is annotated to the resulting image file and is available for display to the user.

In an embodiment the user interface can be used as a tool during install and turnover to the customer, whereby each type of exam (Chest AP nodules, Chest AP fractures, Wrist Lateral, etc.) can be “customized” according to the customer/user preferences.

In an embodiment the user is given an opportunity to update and store various interactions and relationships in the database. The user interface can also be used iteratively/periodically, whereby “feedback” is provided to it in terms of image review and image ratings/rankings. The database can then adapt to this specific customer feedback.

In an embodiment a computer program, provided on one or more computer readable media for selecting plurality of acquisition parameters in tomosynthesis imaging system is provided. The computer program comprising a routine for providing a user interface for allowing a user to specify at least one characteristic of a reconstructed image; and a routine for defining at least one acquisition parameter and processing parameter based on the user-specified image characteristic. The routine for defining at least one of a plurality of acquisition parameter and processing parameters comprises: a routine for obtaining at least one image characteristic from the user, the image characteristics includes characteristics of reconstructed image including slice thickness, ripple artifacts, image noise level, motion artifacts or field-of-view and the anatomic characteristics including body part thickness, high contrast structures that create ripple artifacts, anatomic density or scan orientation. The routine for defining at least one of a plurality of acquisition parameter and processing parameter further comprises: a routine for deriving at least one of acquisition parameters and processing parameters using a data base which stores relations between the image characteristics with acquisition parameters and processing parameters.

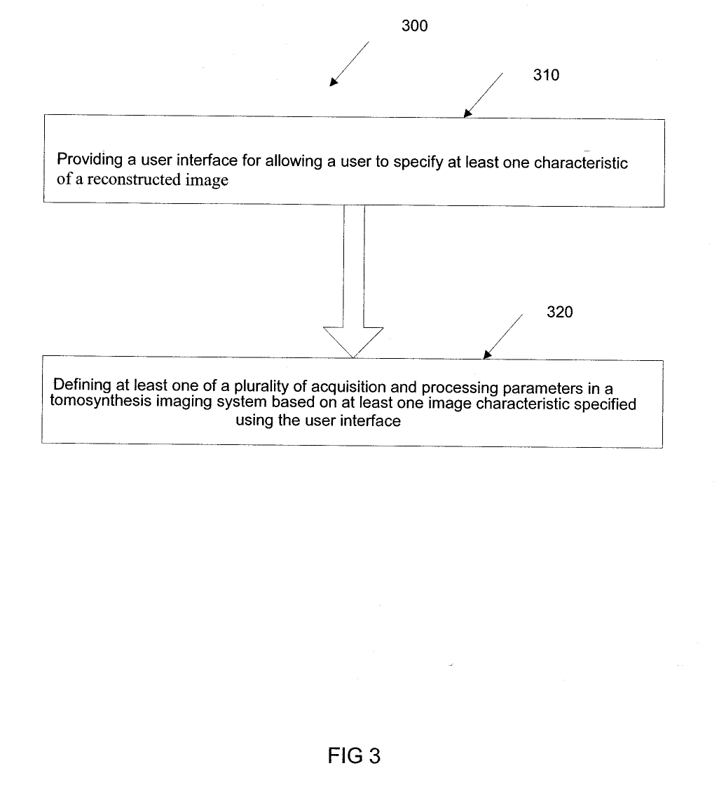

FIG. 3 is a flow chart illustrating the exemplary steps of selecting at least one of desired acquisition and processing parameters as described in an embodiment of the invention. The method of selecting desired at least one of acquisition and processing parameters 300 is explained below: At step 310, a user interface is provided for allowing a user to specify at least one characteristic of a reconstructed image. The user interface is configured to be a visual interface, which will allow the user to select a plurality of image characteristics. The user interacts with the user interface for specifying any of a plurality of image characteristics. The user may specify at least one image characteristic as a specific value or as a value within a range of desired values. Also the user specifies relative importance or significance of a plurality of image characteristics. The image characteristics include characteristics of reconstructed image and anatomic characteristics specific to a patient and an exam. The characteristics of the reconstructed image are selected from a group consisting of slice thickness, ripple artifacts, image noise level, motion artifacts and field-of-view, and anatomic characteristic are selected from a group consisting of body part thickness, high contrast structures, both natural and implanted, that create ripple artifacts, anatomic density and scan orientation. At step 320, at least one of a plurality of acquisition and/or processing parameters is defined based on at least one image characteristic specified by the user using the user interface. The user interface interacts with a processor. The processor is configured to derive one or more acquisition and processing parameters using the user-selected image characteristics. The processor interacts with a database, the data base is configured to store relations between the image characteristics with acquisition parameters and processing parameters. The acquisition parameters includes X-ray source sweep angle, number of projections, dose-per-projection, total dose, X-ray exposure time or collimation and the processing parameter includes reconstruction filter, slice pitch, edge enhancement, noise reduction, number of reconstructed images, averaging or combining of reconstructed images.

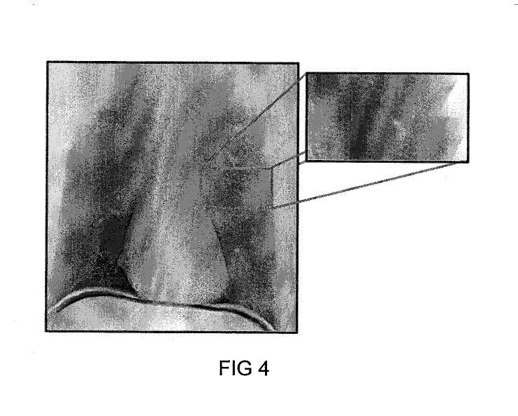

FIG. 4 shows a reconstructed image illustrating the effects of rippling artifacts in an anthropomorphic chest phantom reconstructed image. This illustrates that the ripple artifacts varies greatly based on the anatomy and acquisition parameters.

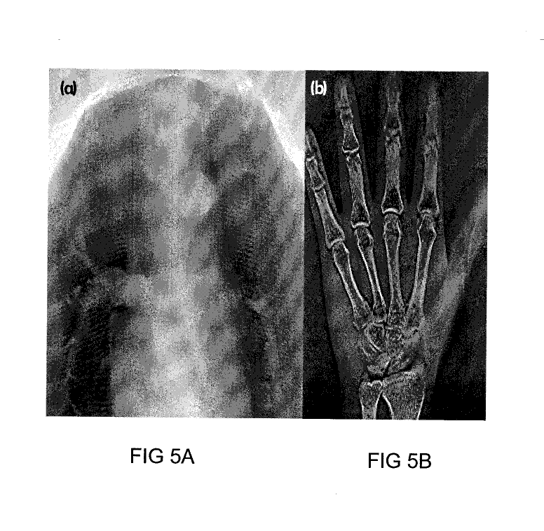

FIGS. 5A and 5B shows reconstructed images depicting the relationship between rippling artifacts and thickness of body part being imaged. The figures illustrate the effect of body part thickness on the impact of ripple artifact through a comparison of a thin hand and a relatively thick chest. FIG. 5A illustrates the reconstructed image of a chest, in which the effects of artifacts are more than that seen in FIG. 5B, which illustrates the image of hand. The figures display the chest image reconstruction and the hand image reconstruction for the same number of projections and sweep angle. The chest image displays extreme rippling artifacts. The hand image, however, shows no ripple artifact. The difference in rippling artifacts in these figures is due to the difference in body part thickness. The hand is not thick enough to suffer from rippling. Ripple artifact level is directly proportional to the body part thickness and the contrast interface. The ripple artifact effects may be controlled by increasing the projection density. Increasing the projection density includes increasing the number of projections and the sweep angle.

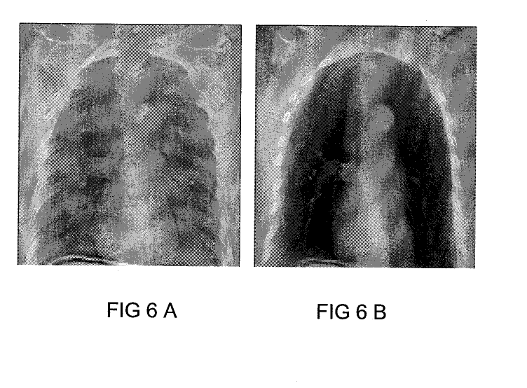

FIGS. 6A and 6B show reconstructed images depicting the relationship between sweep angle and slice thickness. The figures compare the effect of sweep angle on perceived slice thickness in the reconstructed image. FIG. 6A illustrate with the narrow sweep angle (5 degrees) the object is imaged with a relatively thick slice thickness whereas FIG. 6B illustrates with the wider (40 degree) sweep angle, a much thinner plane in the object is imaged. The sweep angle is indirectly proportional to the body part thickness.

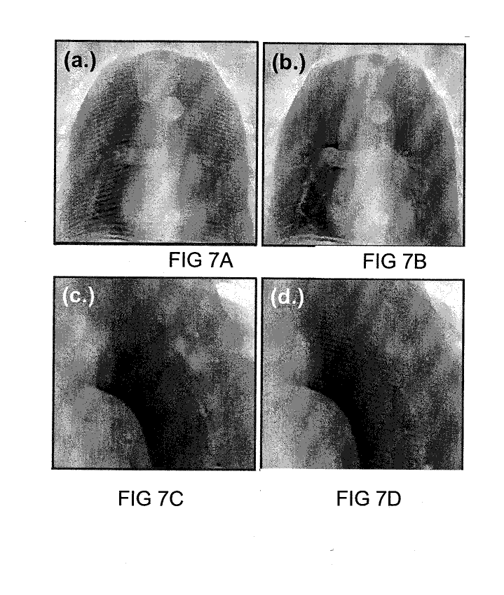

FIGS. 7A, 7B, 7C and 7D show reconstructed images depicting the relationship between projection density, sweep angle and ripple artifacts. The projection density includes number of projections and sweep angle. FIGS. 7A and 7B show reconstructed images depicting the relationship between number of projections and ripple artifacts. FIG. 7A shows a reconstructed image with 10 projections per acquisition and FIG. 7B shows the reconstructed image with 40 projections. Both the images are taken at a 40 degree sweep angle. The figures indicate that the number of projections in an acquisition is inversely proportional to the ripple artifacts.

FIGS. 7C and 7D show reconstructed images depicting the relationship between sweep angle and ripple artifacts. FIG. 7C shows a reconstructed image with 30 degree sweep angle and FIG. 7D show a reconstructed image with 50 degree sweep angle. In both the figures the number of projections are kept at 40. It is seen that that as the sweep angle increase the ripple artifacts reduces. Thus FIGS. 7A to 7D show that projection density of the acquisition is determined based on the desired artifact level.

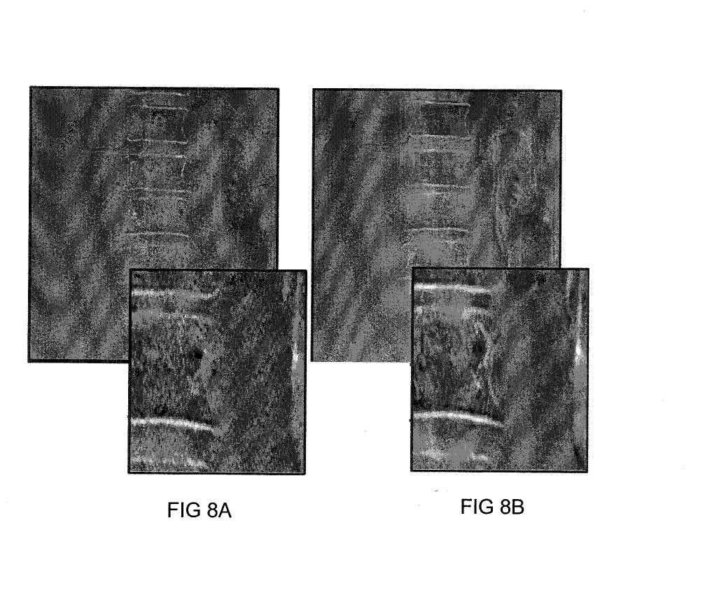

FIGS. 8A and 8B show reconstructed images depicting the relationship between dose of the beam and artifacts. FIG. 8A shows a reconstructed image with a dose of 0.4 mAs/projections and FIG. 8B shows a reconstructed image with 2.0 mAs/Projections. It is clear from the figures that the increase in dose will improve the quality of the image by reducing the noise level. However the dose of the beam intensity is decided by tissue density, patient orientation and body part thickness.

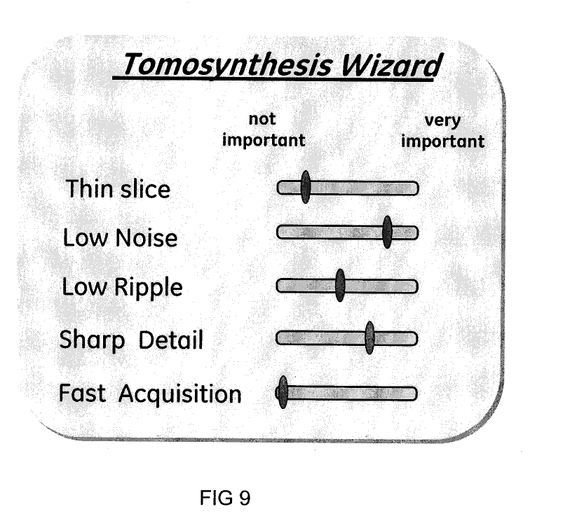

FIG. 9 shows an example of a user interface as described in an embodiment of the invention. The figure shows the visual appearance of the user interface. The user interface allows the user to select the listed characteristics of reconstructed image. The figure is an example of various formats on which the interface can appear. The visual interface can have different interface keys or buttons for selecting image characteristics, anatomic characteristics, acquisition parameters etc.

Thus, various embodiments of this invention provide a method of selecting at least one of a plurality of acquisition and processing parameters in a tomosynthesis imaging system. Further embodiments of this invention provide a tomosynthesis imaging system with enhanced efficiency and reduced complexity.

It should be noted that although the flow charts provided herein show a specific order of method steps, it is understood that the order of these steps may differ from what is depicted. Also, two or more steps may be performed concurrently or with partial concurrence. It is understood that such variations are within the scope of the invention.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

What is claimed is:

1. A method of defining at least one of a plurality of acquisition and processing parameters in a tomosynthesis imaging system, comprising the steps of:
providing a user interface for allowing a user to specify at least one characteristic of a reconstructed image; and
defining at least one of a plurality of acquisition and processing parameters in a tomosynthesis imaging system based on at least one image characteristic specified using the user interface.

2. A method as in claim 1, wherein the user interface is configured to be a visual interface.

3. A method as in claim 1, wherein the user interface is configured to allow a user to specify at least one image characteristic as a specific value or as a value within a range of desired values.

4. A method as in claim 1, wherein the user interface is configured to allow a user to specify a plurality of image characteristics based on their relative importance.

5. A method as in claim 1, wherein the image characteristics include characteristics of reconstructed image and anatomic characteristics specific to a patient and an exam.

6. A method as in claim 5, wherein the characteristics of the reconstructed image are selected from a group consisting of slice thickness, ripple artifact level, image noise level, motion artifacts and field of view, and the anatomic characteristics are selected from a group consisting of body part thickness, high contrast structures, both natural and implanted, that create ripple artifacts, anatomic density and scan orientation.

7. A method as in claim 1, wherein the user interface is further configured for allowing a user to specify at least one of the plurality of the acquisition and processing parameters.

8. A method as in claim 1, wherein the step of defining comprises: interacting the user interface with a processor, the processor being configured to derive at least one of the plurality of acquisition and processing parameters based on the user-specified image characteristics.

9. A method as in claim 9, wherein the step of defining further comprises: interacting the processor with a data base, the data base being configured to store relations between the image characteristics and the acquisition and processing parameters.

10. A method as in claim 8, wherein the step of defining comprises: deriving at least one acquisition parameter based on the user-specified image characteristics, the acquisition parameters include X-ray source sweep angle, number of projections, dose per projection, total dose, X-ray exposure time or collimation.

11. A method as in claim 1, wherein the step of defining comprises: deriving at least one processing parameter based on user-specified image characteristics, the processing parameter includes reconstruction filter, slice pitch, edge enhancement, noise reduction, number of reconstructed images, and averaging or combining of reconstructed images.

12. A method as in claim 1, wherein an input to the user interface is captured and annotated to a resulting image and is available for display to the user.

13. A method as in claim 1, wherein the defined acquisition and processing parameters is annotated to a resulting image file and is available for display to the user.

14. A tomosynthesis system comprising:
an imager for providing images; and
a computer comprising: a user interface for allowing a user to specify at least one characteristic of a reconstructed image; and a processor coupled to the user interface, the processor being programmed to define at least one of a plurality of acquisition and processing parameters for the imager based on at least one user-specified image characteristic.

15. A tomosynthesis system as in claim 14, wherein the processor further comprises a memory configured for storing a database having relations between the image characteristics and the acquisition and processing parameters.

16. A tomosynthesis system as in claim 15, wherein the processor interacts with the database for deriving acquisition and processing parameters based on user-specified image characteristics.

17. A tomosynthesis system as in claim 14, wherein the user interface is a visual interface having a plurality of interface keys for specifying image characteristics, the interface keys being configured to be displayed automatically upon frequent use.

18. A tomosynthesis system as in claim 14, wherein the characteristics of the reconstructed image include slice thickness, ripple artifacts, image noise level, motion artifacts, field of view and the anatomic characteristics including of body part thickness, high contrast structures that creating ripple artifacts, anatomic density or scan orientation.

19. A tomosynthesis system as in claim 14, wherein the acquisition parameters include X-ray source sweep angle, number of projections, dose per projection, total dose, X-ray exposure time or collimation and the processing parameters include reconstruction filter, slice pitch, edge enhancement, noise reduction, number of reconstructed images, and averaging or combining of reconstructed images.

20. A computer program, provided on one or more computer readable media, for selecting at least one of a plurality of acquisition and processing parameters in a tomosynthesis imaging system comprising: a routine for providing a user interface for allowing a user to select at least one characteristic of a reconstructed image; and a routine for defining at least one of a plurality of acquisition and processing parameters based on the user-specified image characteristic.

21. A computer program as claimed in claim 20, wherein the routine for defining comprises: a routine for obtaining at least one characteristic of the reconstructed image from the user, the characteristics of the reconstructed image including slice thickness, ripple artifacts, image noise level, motion artifacts or field-of-view and the anatomic characteristics includes body part thickness, high contrast structures that create ripple artifacts, anatomic density or scan orientation.

22. A computer program as claimed in claim 20, wherein the routine for defining comprises: a routine for defining at least one of plurality of acquisition and or processing parameter based on user-specified characteristic of the reconstructed image, the acquisition parameters includes X-ray source sweep angle, number of projections, dose per projection, total dose, X-ray exposure time or collimation and the processing parameter includes reconstruction filter, slice pitch, edge enhancement, noise reduction, number of reconstructed images, averaging or combining of reconstructed images.

23. A computer program as claimed in claim 20, wherein the routine for defining further comprises: a routine for deriving at least one of acquisition and processing parameters using a data base which stores relations between the image characteristics with acquisition and processing parameters.
